(19) United States
(12) Patent Application Publication
(10) Pub. No.: US 2026/0318844 A1
(43) Pub. Date: Oct. 15, 2026

(54) METHOD FOR PREPARING AND CALIBRATING STANDARD SAMPLE FOR QUANTITATIVE ANALYSIS OF INCLUSIONS IN STEEL AND APPLICATION METHOD

(71) Applicants: NCS Testing Technology CO.,LTD, Beijing (CN); NCS Jiangsu Testing Technology Co.,Ltd., Suzhou (CN)

(72) Inventors: Yunhai JIA, Beijing (CN); Xiaofen ZHANG, Beijing (CN); Liang SHENG, Beijing (CN); Lei YU, Beijing (CN); Miao HE, Beijing (CN); Danyang ZHI, Beijing (CN)

(21) Appl. No.: 19/683,770

(22) Filed: May 21, 2026

(30) Foreign Application Priority Data
Sep. 24, 2025 (CN) 202511374143.3

(51) Int. Cl. G01N 21/892; G01N 1/28; G01N 21/67; G16C 60/00

(52) U.S. Cl. CPC G01N 21/892; G01N 1/286; G01N 21/67; G16C 60/00; G01N 2001/2866; G01N 2021/8925

(57) ABSTRACT

A method for preparing and calibrating a standard sample for quantitative analysis of inclusions in steel and an application method are provided. The method fabricates indentation micropores on the surface of a steel matrix sample, fills nano-oxide powder of the target element to obtain the standard sample, and determines a quantitative relationship model between indentation size and powder mass; and performs emission spectroscopy analysis on the standard sample to obtain the characteristic spectral line intensity of each element at the indentation micropores, and establishes a quantitative calibration curve between elemental content and spectral intensity based on the powder masses corresponding to the indentation micropores. Meanwhile, the present disclosure also discloses the application of the above-mentioned standard sample and the calibration method thereof in the quantitative analysis of non-metallic inclusions in steel.

### CROSS REFERENCE TO RELATED APPLICATIONS

The present disclosure claims the priority to the Chinese patent application with the filing No. 202511374143.3, entitled “PREPARATION AND CALIBRATION OF STANDARD SAMPLE FOR QUANTITATIVE ANALYSIS OF INCLUSIONS IN STEEL AND APPLICATION METHOD” and filed on Sep. 24, 2025 with the Chinese Patent Office, the contents of which are incorporated herein by reference in their entirety.

### TECHNICAL FIELD

The present disclosure relates to the field of metal material inclusion analysis technology, and in particular to a method for preparing and calibrating a standard sample for quantitative analysis of inclusions in steel and an application method.

### BACKGROUND ART

The presence of non-metallic inclusions in steel disrupts the continuity of the steel matrix, adversely affecting the steel's ductility, fatigue resistance, and corrosion resistance. To produce high-quality steel with higher purity, it is necessary not only to continuously improve inclusion control processes but also to develop more accurate and efficient inclusion detection and quantitative analysis techniques. In recent years, spectroscopic inclusion composition detection technologies have developed rapidly, with methods such as Laser-Induced Breakdown Spectroscopy (LIBS), Spark Spectroscopy combined with Original Position Statistic Distribution Analysis (OPA), Optical Emission Spectroscopy combined with Pulse Distribution Analysis (OES-PDA), and in-site Spark Mapping Analysis for Large Samples (SMALS) being widely applied. However, due to the lack of commercially available non-metallic inclusion standard samples for reference, it is impossible to directly and accurately correlate spectral signal intensity with the chemical composition or type of inclusions, leading to the following widespread common problems of existing spectroscopic analysis methods:
it relies on inclusion particle size data obtained by scanning electron microscopy-energy dispersive spectroscopy (SEM-EDS) as the basis for modeling, and cannot achieve independent measurement;
the quantitative characterization ability of complex inclusions is insufficient, and due to their complex structure and multi-component interference, existing studies are mostly limited to single types of inclusions;
the entire sample surface needs to be scanned point by point to accumulate a large amount of data, which is a tedious, time-consuming and labor-intensive process; and
ultimately, only the size information of the inclusions can be obtained, which cannot accurately reflect their content.

Therefore, developing a series of non-metallic inclusion standard samples with controllable content and covering multiple elements and chemical concentration gradients is a key prerequisite for achieving accurate spectroscopic quantitative analysis. However, the preparation of steel-based standard samples with controllable inclusion content is technically extremely difficult, and effective methods are currently lacking. In particular, non-metallic inclusions in steel are diverse in type and have complex physicochemical properties, often exhibiting irregular and difficult-to-identify morphological features under an optical microscope. These inclusions typically have complex chemical compositions, containing multiple metallic and non-metallic elements, resulting in severe signal interference; their particle size distribution is concentrated, lacking a size gradient suitable for calibration; their morphology is irregular, with large aspect ratio differences and unstable geometric forms, making accurate identification and quantification difficult to be achieved through conventional image analysis methods. Therefore, how to prepare standard samples containing inclusions to further achieve quantitative analysis of more complex inclusions with different elemental compositions has become a technical problem to be urgently solved in the art.

### SUMMARY

The purpose of the present disclosure is to provide a method or preparing and calibrating a standard sample for quantitative analysis of inclusions in steel and an application method. By preparing a standard sample containing non-metallic inclusions, an inclusion content standard curve can be established quickly and independently without the need for SEM-EDS, providing technical support for the spectral content detection of diatomic inclusions and complex multi-element inclusions.

To achieve the above purpose, the present disclosure provides the following solutions:
a method for preparing and calibrating a standard sample for quantitative analysis of inclusions in steel, the method comprising the following steps:
preparation of the standard sample: fabricating indentation micropores on the surface of a steel matrix sample, filling nano-oxide powder of a target element to obtain the standard sample, and determining a quantitative relationship model between indentation size and powder mass; and
calibration curve establishment: performing emission spectroscopy analysis on the standard sample to obtain the intensity of the characteristic spectral line of each element at the indentation micropores, and establishing a quantitative calibration curve between elemental content and spectral intensity based on the powder mass corresponding to the indentation micropores.

Further, the fabricating indentation micropores on the surface of a steel matrix sample and filling a target element nano-oxide powder to obtain the standard sample specifically includes:
S1, micropore array preparation: applying a gradient load on the surface of the polished steel matrix sample by using a Vickers hardness tester to form a series of square pyramidal indentation micropores with different sizes;
S2, powder filling and fixation: placing the steel matrix sample with the indentation micropores in a container containing nano-oxide powder of the target element, and vacuuming and then using a cold isostatic pressing technology to make the powder densely filled in the indentation micropores.
S3, surface treatment: grinding the surface of the steel matrix sample to remove excess powder, to ensure that only the powder filled in the indentation micropores is retained to obtain the standard sample with a smooth surface.

Further, the determining a quantitative relationship model between indentation size and powder mass specifically includes:
S4, quantitative characterization and calibration: using a white light interferometer to measure three-dimensional morphology of the indentation micropores to obtain the volume of each indentation micropore and the contained powder mass derived based on the volume and the theoretical powder density, and establishing the quantitative relationship model between indentation size and powder mass.

Further, the micropore array preparation of S1 specifically includes:
selecting multiple regions on the surface of the polished steel matrix sample, applying a series of gradient loads by using the Vickers hardness tester, and maintaining the pressure (load) for a certain period of time and then unloading, where the magnitudes of the gradient loads are designed based on the laser beam spot size for the emission spectroscopy analysis and the common particle size distribution range of the target detection inclusion in the steel; and
fabricating an indentation array with a set number of rows and columns on the surface of a steel matrix sample to form the series of square pyramidal indentation micropores with different sizes, and determining the spacing between the indentation micropores based on the laser beam spot size for the emission spectroscopy analysis, such that the beam spot does not affect the adjacent indentation micropore regions during laser excitation.

Further, the powder filling and fixing of S2 specifically includes:
placing the steel matrix sample with the indentation micropores in the container containing the nano-oxide powder of the target element, where the container is a flexible rubber mold, the nano-oxide powder of the target element is a single-component powder or a multi-component composite powder, and the multi-component composite powder is prepared by weighing and uniformly mixing multiple element powders in a predetermined ratio;
preparing, if the single-component powder is filled, multiple indentation micropore samples with a series of gradient loads, and pressing nano-oxide powders of different elements thereinto respectively, to thereby obtain multiple univariate standard samples with different concentration gradients;
preparing, if the multi-component composite powder is filled, an indentation micropore array sample with the same load, and before filling, weighing the mass of each component and calculating the ratio thereof to obtain a multivariate standard sample that can obtain the concentration gradient relationship of multiple elements at one time; and
sealing the mold filled with powder, connecting to a high vacuum system for vacuuming treatment; then placing the sealed mold in a cold isostatic press, and applying hydrostatic pressure at room temperature and holding the pressure for a certain period of time, where during this process, the isotropic hydrostatic pressure causes the nano-oxide powder of the target element to fill the square pyramidal cavity of each indentation micropore evenly and densely, to form a standard detection point with clear composition and controllable content.

Further, the quantitative relationship model between indentation size and powder mass is expressed as follows:

$$C ( wt \% ) = \frac{{W}_{A}}{{W}_{max}} = \frac{1 / 3 \cdot {S}_{A} \cdot {h}_{A} \cdot {ρ}_{A}}{1 / 3 \cdot {S}_{max} \cdot {h}_{max} \cdot {ρ}_{A}} = \frac{{V}_{A}}{{V}_{max}}$$

where W_{A} represents the absolute mass of the nano-oxide powder of the target element filled in an indentation micropore, Wₘₐₓ represents the powder mass in the largest indentation micropore, and the largest indentation represents the maximum sample amount that the system can analyze, which is used as a baseline to establish relative mass ratio relationship, and the volume geometric parameters of each gradient indentation are converted through formulaic calculation into precise mass percentages, which both eliminates the uncertainty of absolute mass measurement and ensures the linear relationship of the concentration gradient; ρ_{A} represents the theoretical density of the nano-oxide powder of the target element; V_{A} represents the volume of the nano-oxide powder (indentation) of the target element; Vₘₐₓ represents the volume of the largest indentation micropore; S_{A} represents the bottom area of the nano-oxide powder (indentation) of the target element, and Sₘₐₓ represents the bottom area of the largest indentation; and C(wt %) representing the content (relative mass percentage) of the nano-oxide powder of the target element.

Further, the performing emission spectroscopy analysis on the standard sample specifically includes:
S5, using LIBS or Spark-OES targeted analysis technology to perform positioned excitation on the indentations on the standard sample to collect the intensity of the characteristic spectral line of each element, which includes the following steps:
placing the prepared standard sample in a laser-induced breakdown spectroscopy or spark-source emission spectroscopy analysis system, and scanning and observing the surface of the standard sample by using the high-precision moving platform and microscopic positioning device equipped in the system, to locate each indentation micropore region filled with the nano oxide powder of the target element;
formulating corresponding laser excitation strategies based on the differences in the sizes of the indentation micropores, where for indentation micropores with a size larger than the set value, a multi-pulse laser excitation mode is adopted; and for indentation micropores with a size smaller than the set value, a single-pulse laser excitation mode is selected; and
after excitation at each positioning point, acquiring the original characteristic spectral signal of the target element, and then processing the original characteristic spectral signal by a continuous background subtraction algorithm to eliminate matrix interference and obtain the net intensity value of the characteristic spectral line of each element at the indentation micropores.

Further, the establishing the quantitative calibration curve between elemental content and spectral intensity is specifically as follows:
S6, establishing the quantitative calibration curve between elemental content and spectral intensity based on the powder mass and the measured net spectral intensity, which includes the following steps:
performing linear fitting in a double logarithmic coordinate system, by using the powder masses corresponding to the indentation micropores calculated based on the quantitative relationship model between indentation size and powder mass as the ordinate, and the measured intensity of the characteristic spectral line of each element at the indentation micropores as the abscissa, to obtain the quantitative calibration curve of the element.

The present disclosure also provides a standard sample, which is obtained by the method for preparing and calibrating a standard sample for quantitative analysis of inclusions in steel as described above. The standard sample is a univariate standard sample or a multivariate standard sample.

The present disclosure further provides an application of the method for preparing and calibrating a standard sample for quantitative analysis of inclusions in steel, which is specifically a method for quantitative analysis of multiple non-metallic inclusions in steel based on emission spectroscopy, including the following steps:
obtaining a quantitative calibration curve between elemental content and spectral intensity by applying the above-mentioned method for preparing and calibrating a standard sample for quantitative analysis of inclusions in steel; or, establishing the quantitative calibration curve between elemental content and spectral intensity by using the above-mentioned standard sample; and
performing emission spectroscopy analysis on a to-be-tested steel sample to obtain the intensity of the characteristic spectral line of each element in the inclusions, and quantitatively analyzing the elemental content of the inclusions in the to-be-tested steel sample based on the quantitative calibration curve between elemental content and spectral intensity.

According to specific embodiments provided by the present disclosure, the present disclosure discloses the following technical effects. The method for preparing and calibrating a standard sample for quantitative analysis of inclusions in steel provided by the present disclosure involves fabricates a Vickers indentation micropore array on the surface of a steel matrix, fills specific nano-oxide powder therein, and forms a standard sample containing non-metallic inclusions with known and controllable content after cold isostatic pressing and precision grinding. Further, the three-dimensional morphology of the indentation is characterized using a white light interferometer, etc., to accurately calculate the mass of the filled powder, and the laser-induced breakdown spectroscopy (LIBS) or spark-source emission spectroscopy (Spark-OES) targeted analysis technology is used to establish a quantitative calibration curve of elemental spectral intensity versus content, which can be directly applied to the quantitative analysis of the elemental content of inclusions in the to-be-tested steel sample.

The present disclosure solves the problem of lack of standard samples when quantitatively analyzing non-metallic inclusions in steel by using emission spectroscopy, can accurately establish standard curves for the quantitative analysis of diatomic non-metallic inclusions and multi-element composite inclusions without relying on pre-calibration with scanning electron microscope-energy dispersive X-ray spectrometer (SEM-EDS), and has the advantages of high precision and traceability.

### BRIEF DESCRIPTION OF DRAWINGS

To more clearly illustrate the technical solutions in the embodiments of the present disclosure or the prior art, the drawings required to be used in the embodiments will be briefly introduced below. Obviously, the drawings described below are only some embodiments of the present disclosure. For those skilled in the art, other drawings may be obtained based on these drawings without creative effort.

FIG. 1 is a flowchart of a method for preparing and calibrating a standard sample for quantitative analysis of inclusions in steel according to the present disclosure;

FIG. 2 is a schematic diagram of the formation of a square pyramidal indentation micropore on a steel matrix by a Vickers hardness tester indenter provided by the present disclosure;

FIG. 3 is a schematic diagram of the indentation size distribution corresponding to the gradient load provided in embodiments of the present disclosure;

FIG. 4 is a schematic diagram of the preparation process of the standard sample with controllable inclusion content provided in embodiments of the present disclosure; and

FIG. 5 is a three-dimensional morphology diagram of the indentation after powder filling, characterized by the white light interferometer, provided in embodiments of the present disclosure.

### DETAILED DESCRIPTION OF EMBODIMENTS

The technical solutions of the embodiments of the present disclosure will be clearly and completely described below with reference to the drawings. Obviously, the described embodiments are only some embodiments of the present disclosure, and not all embodiments. Based on the embodiments of the present disclosure, all other embodiments obtained by those ordinarily skilled in the art without creative effort are within the scope of protection of the present disclosure.

The present disclosure proposes a method for preparing an inclusion standard sample with controllable content for different elements, which performs accurate positioning and detection on the non-metallic inclusions in steel by an emission spectroscopy platform equipped with a three-dimensional high-precision motion platform system and an image acquisition system to obtain the characteristic spectral line intensities of target elements within the inclusions. By correlating the elemental content and spectral line intensities in various inclusions, the quantitative curve is established, enabling quantitative analysis of more complex inclusions with different elemental compositions. The present disclosure aims to solve the problem that in the process of quantifying inclusions in a steel matrix by spectroscopic methods, there are no readily available inclusion standard samples, and quantitative analysis can only be performed after obtaining inclusion size data through preliminary experiments using SEM-EDS instruments and then combining with statistical data on spectral intensities to establish a standard curve. The method described in the present disclosure allows for the rapid and independent establishment of inclusion content standard curves without the need for SEM-EDS, providing a foundation for the spectral content detection of diatomic inclusions and complex multi-element inclusions.

To make the above-mentioned purposes, features and advantages of the present disclosure more apparent and understandable, the present disclosure will be further described in detail below with reference to the drawings and specific embodiments.

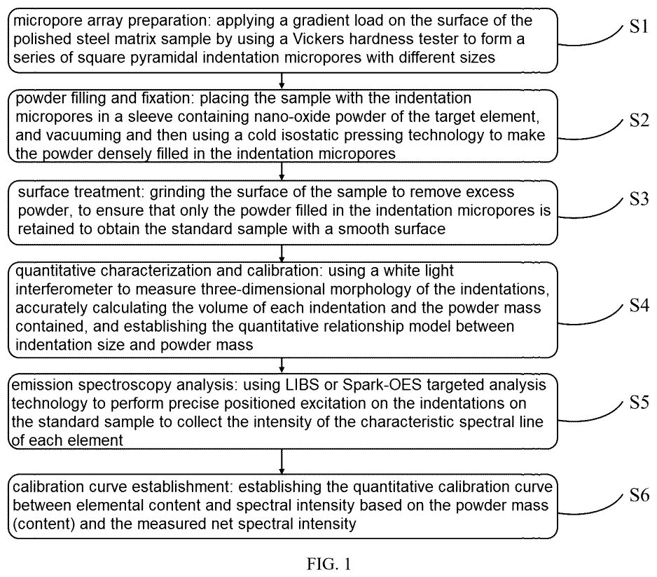

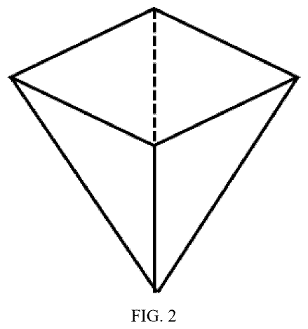

As shown in FIG. 1, the method for preparing and calibrating a standard sample for quantitative analysis of inclusions in steel provided by the present disclosure includes the following steps:
S1, micropore array preparation: applying a gradient load on the surface of the polished steel matrix sample by using a Vickers hardness tester to form a series of square pyramidal indentation micropores with different sizes, as shown in FIG. 2;
S2, powder filling and fixation: placing the steel matrix sample with the indentation micropores in a container containing nano-oxide powder of the target element, and vacuuming and then using a cold isostatic pressing technology to make the powder densely filled in the indentation micropores;
S3, surface treatment: grinding the surface of the steel matrix sample to remove excess powder, to ensure that only the filler in the indentation micropores is retained to obtain the standard sample with a smooth surface;
S4, quantitative characterization and calibration: using a white light interferometer to measure three-dimensional morphology of the indentation, accurately calculating the volume of each indentation and the powder mass contained therein, and establishing a quantitative relationship model between indentation size and powder mass;
S5, emission spectroscopy analysis: using LIBS or Spark-OES targeted analysis technology to perform precise positioned excitation on the indentations on the standard sample to collect the intensity of the characteristic spectral line of each element; and
S6, calibration curve establishment: establishing the quantitative calibration curve between elemental content and spectral intensity based on the powder mass (content) and the measured net spectral intensity.

Specifically, the micropore array preparation in step S1 specifically includes the following contents.

In S101, to form a set of standard points with content gradients, multiple regions are selected on the matrix surface during sample preparation, and a series of gradient loads are systematically applied. The specific magnitude of the applied load can be adjusted according to the laser beam size of the spectral analysis method used, and at the same time, is designed with reference to the common particle size distribution range of the target detection inclusions in steel. The size of the Vickers indentation can be flexibly controlled to approximate the typical size of actual inclusions at the microscale, thereby better simulating the analytical scenario of real samples.

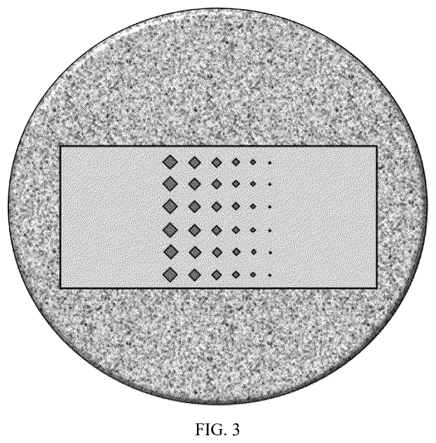

In S102, an indentation array with a certain number of rows and columns can be planned and fabricated on the sample surface. The specific scale may be determined according to actual working needs to ensure the statistical reliability of the data. The spacing between the indentations is also flexibly set, and mainly adjusted according to the size of the spectral beam spot, to ensure that the beam spot does not affect adjacent indentation regions during subsequent laser excitation, avoiding signal cross-contamination. At the same time, sufficient spacing can also effectively avoid stress interference between indentations during sample preparation. The indentation size distribution is as shown in FIG. 3.

In S103, the sample itself is not a disposable consumable; its size may be selected according to actual working needs. Larger samples allow for the fabrication of more indentation arrays, significantly increasing the number of uses of a standard sample from a single preparation, thus improving the efficiency and practicality of the method. After applying the load, the pressure (load) is maintained for a certain period of time and then unloaded to form a stable regularly-shaped square pyramidal indentation. By systematically designing the load gradient, it can be ensured that the equivalent diameter range of the final obtained indentations not only matches the ablation characteristics of the spectral beam spot well, but also covers a wide dynamic content range from trace amount to near-complete filling, laying a solid foundation for subsequently establishing of accurate and reliable quantitative calibration curves.

Specifically, the powder filling and fixation in step S2 specifically includes the following contents.

In S201, the matrix sample with the indentation array is carefully placed into a flexible rubber mold (or called container), and then an appropriate amount of high-purity nano-oxide powder of the target element is added into the mold. The nano-oxide powder may be a single component or multi-component composite powder prepared by accurately weighing and uniformly mixing multiple powders in a predetermined ratio. The sample prepared by filling the single-component powder is called a univariate inclusion standard sample; and the sample prepared by filling mixed powder is called a multivariate inclusion standard sample. Both may be used to analyze single inclusions or multi-element composite inclusions. Users may choose to prepare the univariate or multivariate standard sample according to their actual testing needs.

In S202, if the single powder is used, multiple indentation micropore samples with a series of gradient loads need to be prepared, and nano-powders of different elements are pressed into them respectively, so as to obtain multiple univariate standard samples with different concentration gradients.

In S203, if the mixed powder is used, it is not necessary to prepare multiple indentation micropore samples with a series of gradient loads; only one indentation micropore array sample with the same load needs to be fabricated. The mass of each component must be accurately weighed and the ratio thereof calculated before filling. This data is used as known parameter for subsequent calculation of the total mass of the filler in the indentation and the content of each component, and used to plot the standard curve. The multivariate standard sample can obtain the concentration gradient relationships of multiple elements at one time, significantly improving analytical efficiency.

In S204, the mold filled with powder is sealed and then connected to a high vacuum system for vacuuming treatment to completely eliminate gas in the indentation micropores and the gaps in the interstices of the powder, preventing residual gas from affecting the dense filling effect of the powder during subsequent pressurization. Next, the sealed mold is placed in a cold isostatic press, where a high hydrostatic pressure is applied at room temperature and maintained for a certain period of time. During this process, the isotropic hydrostatic pressure promotes the uniform and dense filling of the nano-powder into the square pyramidal cavity of each indentation, thereby forming standard detection points with clear composition and controllable content.

Specifically, the quantitative characterization and calibration in step S4 specifically includes the following contents.

The surface of the prepared standard sample is scanned using a white light interferometric surface profiler. Three-dimensional morphology measurements were performed on multiple indentations at each load level to obtain high-precision information on the indentation cavity height, to check whether the indentation cavity was densely filled with powder and the surface was smooth. By using the instrument's accompanying analysis software, the volume V of each square pyramidal indentation can be accurately calculated. Knowing the theoretical density p of the nano-powder used, the absolute mass W of the nano-powder filled in each indentation can be accurately calculated. For quantitative calibration, the powder mass in the largest indentation was typically set as the baseline (100%), and the powder mass in other indentations is converted to its relative mass percentage. Thus, a precise and traceable quantitative relationship model is established for each indentation, from a fabrication parameter (load) to geometric dimension (volume) to physical quantity (mass):

$$C ( wt \% ) = \frac{{W}_{A}}{{W}_{max}} = \frac{1 / 3 \cdot {S}_{A} \cdot {h}_{A} \cdot {ρ}_{A}}{1 / 3 \cdot {S}_{max} \cdot {h}_{max} \cdot {ρ}_{A}} = \frac{{V}_{A}}{{V}_{max}}$$

where W_{A} represents the absolute mass of the nano-oxide powder of the target element filled in an indentation micropore, Wₘₐₓ represents the powder mass in the largest indentation micropore, and the largest indentation represents the maximum sample amount that the system can analyze, which is used as a baseline to establish relative mass ratio relationship, and the volume geometric parameters of each gradient indentation are converted through formulaic calculation into precise mass percentages, which both eliminates the uncertainty of absolute mass measurement and ensures the linear relationship of the concentration gradient; ρ_{A} represents the theoretical density of the nano-oxide powder of the target element; V_{A} represents the volume of the nano-oxide powder (indentation) of the target element; Vₘₐₓ represents the volume of the largest indentation micropore; S_{A} represents the bottom area of the nano-oxide powder (indentation) of the target element, and Sₘₐₓ represents the bottom area of the largest indentation; and C(wt %) represents the content (relative mass percentage) of the nano-oxide powder of the target element.

Specifically, the emission spectroscopy analysis in step S5 specifically includes the following contents.

The prepared standard sample is placed in a laser-induced breakdown spectroscopy (LIBS) or spark-source emission spectroscopy (Spark-OES) analysis system. The surface of the sample is scanned and observed by using the high-precision moving platform and microscopic positioning device equipped in the system, to precisely locate each indentation region filled with nano-powder. A corresponding laser excitation strategy is formulated based on the differences in indentation size: for indentations with larger size, a multi-pulse laser excitation mode is used to ensure sufficient excitation of the powder within the indentation cavity; and for indentations with smaller size, a single-pulse laser excitation mode is selected to ensure excitation efficiency while avoiding signal saturation or overlap. After excitation at each positioning point, the characteristic spectral signal (e.g., the Al(I) 396.15 nm spectral line) of the target element is acquired. Subsequently, a continuous background subtraction algorithm is used to process the original spectral signal, eliminating matrix interference and obtaining the net intensity value of the target spectral line for subsequent quantitative analysis.

Specifically, the establishing the quantitative calibration curve between elemental content and spectral intensity based on the powder mass (content) and the measured net spectral intensity in step S6 specifically includes:
performing linear fitting in a double logarithmic coordinate system, by using the powder mass (converted to weight percentage) calculated in step S4 as the ordinate (Y-axis) and the measured net spectral intensity as the abscissa (X-axis), to obtain the quantitative calibration curve for this element. This calibration curve can be directly used for the quantitative analysis of inclusions of the same element type in the unknown steel sample.

The standard samples prepared by the above method can be used for quantitative analysis of univariate inclusions or composite inclusions in steel using laser-induced breakdown spectroscopy or other spectroscopic methods.

### Example 1

In one specific embodiment, the present disclosure provided a method for preparing and calibrating a standard sample for quantitative analysis of multiple non-metallic inclusions in steel by using emission spectroscopy, including the following steps.

A high-purity ultra-low-carbon steel with extremely low inclusion content was selected as the matrix material. The matrix material was cut into a block sample of 20 mm×20 mm×5 mm. The sample was ground and polished using standard metallographic sample preparation procedures, with coarse grinding and fine grinding performed sequentially using diamond grinding discs of 400 grit, 800 grit, 1500 grit, and 2000 grit, and finally, mirror polishing performed using diamond polishing paste with a particle size of 0.3 μm, to ensure a scratch-free, mirror-like surface, thereby obtaining a clear and consistent indentation morphology.

The polished sample was fixed on the sample stage of a micro Vickers hardness tester, and a 136° diamond square pyramid indenter was used. FIG. 2 shows the typical morphology of the formed square pyramidal indentation. This example selected to prepare a univariate standard sample. To form a set of standard points with content gradients, multiple regions were selected on the sample surface, and six gradient loads of HV 0.3, HV 0.2, HV 0.1, HV 0.05, HV 0.025, and HV 0.01 were applied thereto respectively. A 6×6 indentation array was fabricated on the sample surface to ensure statistical reliability. A schematic diagram of the indentation micropore array is as shown in FIG. 3. The indentation spacing was set to be 5 times or more the diagonal length of the indentation, approximately 150-200 μm, to avoid stress interference between indentations. After applying the load, the pressure was held for 15 seconds and then unloaded. The equivalent diameter range thereof was approximately 7 μm to 40 μm.

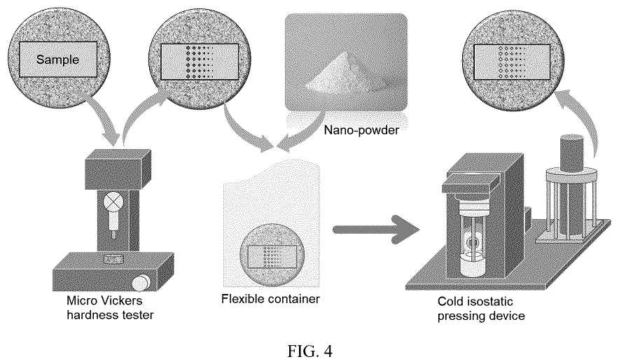

The sample with the indentation array was carefully placed into a flexible rubber container, and a sufficient amount of high-purity Al₂O₃ nano-powder (purity >99.99%, average particle size ≈20 nm) was added into the container. After sealing the mold, it was connected to a high vacuum system, vacuumed to 1×10⁻³ Pa and maintained for 30 minutes to completely remove air in the indentation micropores and the gaps in the interstices of the powder, preventing it from affecting the dense filling of the powder during subsequent pressurization. Subsequently, the entire vacuum-sealed mold was placed in a cold isostatic press and held at a pressure of 200 MPa for 30 minutes at room temperature. During this process, the isotropic hydrostatic pressure uniformly and densely pressed the nano-powder into the square pyramidal cavity of each indentation (see FIG. 4 for a schematic diagram of the process).

After the cold isostatic pressing, the sample was taken out from the mold, and a pressed Al₂O₃ compact layer adhered to the surface thereof and covers the metal surface. A 3000-grit ultrafine diamond sandpaper was used to gently and evenly polish the sample surface in a single direction. The key to this step was to remove only the excess powder layer from the surface while completely preserving the powder already filled within the indentation cavity. Microscopic observation continues until the matrix surface outside the indentation was restored to a mirror finish and no residual powder remained, thus completing the treatment.

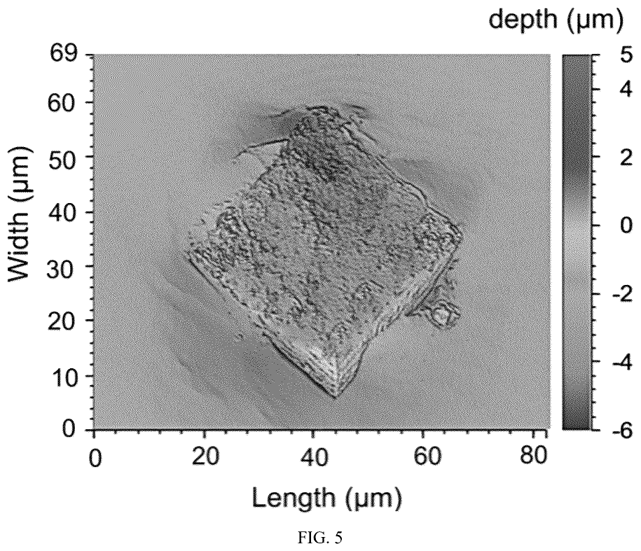

The surface of the prepared standard sample was measured using a white light interferometric surface profiler. Three-dimensional morphology measurement was performed on multiple indentations at each load level to obtain high-precision information on the height of the indentation micropores. FIG. 5 shows a typical three-dimensional morphology diagram of the indentation after filling, demonstrating that the indentation cavity has been densely filled with powder and the surface is smooth.

By using the instrument's accompanying analysis software, the volume V of each square pyramidal indentation can be accurately calculated, thus precisely calculating the absolute mass W of the Al₂O₃ powder filled in each indentation. According to the formula:

$$C ( wt \% ) = \frac{{W}_{A}}{{W}_{max}} = \frac{1 / 3 \cdot {S}_{A} \cdot {h}_{A} \cdot {ρ}_{A}}{1 / 3 \cdot {S}_{max} \cdot {h}_{max} \cdot {ρ}_{A}} = \frac{{V}_{A}}{{V}_{max}}$$

For quantitative calibration, the powder mass within the largest indentation (HV 0.3) is typically set as the baseline (100%), and the powder mass in other indentations is converted to its relative mass percentage. A precise and traceable quantitative relationship model is thereby established for each indentation, from fabrication parameter (load) to geometric dimension (volume) to physical quantity (mass).

The prepared standard sample was placed in a LIBS or Spark-OES analyzer equipped with a high-precision three-dimensional moving platform and a microscopic vision positioning system. Each powder-filled indentation was found and precisely positioned using a microscope camera. Based on the laser ablation characteristics measured in previous experiments (single-pulse ablation pit diameter of approximately 40 μm, depth of approximately 3.5 μm; double-pulse ablation pit diameter of approximately 47 μm, depth of approximately 6.6 μm), a graded excitation strategy was formulated: for large indentations of the levels of HV 0.3, HV 0.2, and HV 0.1, double-pulse laser excitation was used to ensure that laser can complete ablate all powder within the indentation. For small indentations of e levels of HV 0.05, HV 0.025, and HV 0.01, single-pulse laser excitation was used to avoid signal saturation or excessive ablation of the matrix.

The present disclosure utilized the picosecond laser PL2250 from EKSPLA company, Lithuania. The laser beam was Q-switched Nd: YAG laser with pulse output at two operating wavelengths: 1064 nm and 532 nm. To obtain a smaller beam diameter, 532 nm was chosen for the experiment. The beam divergence was 0.5 mrad, the pulse duration was 30 ps, the pulse width was 30 ps, and the energy was 3.8 mJ. A focusing lens with a focal length of 75 mm was used to perpendicularly focus the laser beam onto the sample surface to generate light, resulting in a focused spot diameter of approximately 40 μm. The spectrometer has a spectral range of 140 nm-670 nm, a diffraction grating of 2700 lines/mm @ 500 mm, and a resolution of 0.07 nm. The laser and spectrometer are synchronously controlled by a digital pulse generator, and the spectrometer is triggered by the Q-switching signal from the laser. The experiment was conducted in an air environment with atmospheric pressure, a temperature of 20° C., and an ambient relative humidity of 25%. The spectral intensity of the characteristic atomic line Al(I) of the Al element at 396.15 nm was collected. Ten spectral signals were collected for each indentation, and the average value was taken. The net spectral intensity value I at that point was obtained using a continuous background subtraction algorithm. The calculation formula is as follows:

I(t)−Iₒᵣᵢ(t)−I_{bg}(t)

In the formula, Iₒᵣᵢ is the original spectral intensity, and I_{bg} is the continuous background intensity.

A linear regression fitting was performed using the calculated mass percentage of Al₂O₃ in each indentation (logarithm, lg(Content)) as the ordinate (Y-axis) and the measured net spectral intensity (logarithm, lg(Intensity)) as the abscissa (X-axis). The data points showed a good linear relationship, and a quantitative calibration curve for Al element was successfully established. The mathematical expression of this curve was: lg(Content)=k*lg(Intensity)+b, where k and b were the fitting coefficients.

### Example 2

The calibration curve established using the method described in Example 1 can be used for LIBS or Spark-OES quantitative analysis of Al₂O₃ inclusions found in an unknown steel sample by microscopic observation.

The present disclosure provided a method for quantitative analysis of multiple non-metallic inclusions in steel based on emission spectroscopy, including the following steps.

Emission spectroscopy analysis was performed on the steel sample to be tested. The inclusion was located by the LIBS or Spark-OES system. After excitation, the net intensity of the Al(I) 396.15 nm line was obtained, and substituted into the calibration curve obtained in Example 1, then the approximate content of Al₂O₃ in the inclusion can be back-calculated, thus achieving quantitative analysis.

In summary, the method or preparing and calibrating a standard sample for quantitative analysis of inclusions in steel and an application method provided by the present disclosure can be used for the preparation and calibration of the standard sample for quantitative analysis of multiple non-metallic inclusions in steel using emission spectroscopy. A series of square pyramidal indentation micropores of different sizes are formed by using a Vickers hardness tester to apply a gradient load to the surface of a polished steel matrix sample. The sample with the indentation micropores is placed in a container containing nano-oxide powder of the target element. After vacuuming, cold isostatic pressing is used to densely fill the powder into the indentation micropores. The three-dimensional morphology of the indentation is measured using a white light interferometer, and the volume of each indentation and powder mass contained therein are accurately calculated to establish a quantitative relationship model between indentation size and powder mass. LIBS or Spark-OES targeted analysis technology is used to perform precise positioned excitation on the indentations on the standard sample, and the intensity of the characteristic spectral line of each element is collected. The mass percentage of the prepared inclusion standard sample is correlated with the measured net spectral intensity to establish a quantitative calibration curve between elemental content and spectral intensity, realizing rapid and accurate quantitative spectral analysis of univariate inclusions and multi-element composite inclusions in steel.

The principles and implementation methods of the present disclosure are illustrated using specific examples herein. The descriptions of the above embodiments are only for the purpose of helping to understand the method and core ideas of the present disclosure. Furthermore, those skilled in the art will recognize that, based on the ideas of the present disclosure, there will be changes in the specific implementation methods and application scope. Therefore, the content of this specification should not be construed as a limitation of the present disclosure.

1. A method for preparing and calibrating a standard sample for quantitative analysis of inclusions in steel, comprising following steps:
preparation of the standard sample: fabricating indentation micropores on a surface of a steel matrix sample, filling nano-oxide powder of a target element to obtain the standard sample, and determining a quantitative relationship model between indentation size and powder mass; and
calibration curve establishment: performing emission spectroscopy analysis on the standard sample to obtain intensity of a characteristic spectral line of each element at the indentation micropores, and establishing a quantitative calibration curve between elemental content and spectral intensity based on powder masses corresponding to the indentation micropores.

2. The method for preparing and calibrating a standard sample for quantitative analysis of inclusions in steel according to claim 1, wherein the fabricating indentation micropores on the surface of the steel matrix sample and filling nano-oxide powder of the target element to obtain the standard sample comprises:
S1, micropore array preparation: applying a gradient load on a surface of a polished steel matrix sample by using a Vickers hardness tester to form a series of square pyramidal indentation micropores with different sizes;
S2, powder filling and fixation: placing the steel matrix sample with the indentation micropores in a container containing the nano-oxide powder of the target element, vacuuming, and then using a cold isostatic pressing technology to make the powder densely filled in the indentation micropores; and
S3, surface treatment: grinding the surface of the steel matrix sample to remove excess powder, to ensure that only the powder filled in the indentation micropores is retained to obtain the standard sample with a smooth surface.

3. The method for preparing and calibrating a standard sample for quantitative analysis of inclusions in steel according to claim 2, wherein the determining the quantitative relationship model between indentation size and powder mass comprises:
S4, quantitative characterization and calibration: using a white light interferometer to measure three-dimensional morphology of the indentation micropores to obtain a volume of each of the indentation micropores and contained powder mass derived based on the volume and a theoretical powder density, and establishing the quantitative relationship model between indentation size and powder mass.

4. The method for preparing and calibrating a standard sample for quantitative analysis of inclusions in steel according to claim 2, wherein the micropore array preparation of S1 comprises:
selecting multiple regions on the surface of the polished steel matrix sample, applying a series of gradient loads by using the Vickers hardness tester, and maintaining a pressure for a certain period of time and then unloading, wherein magnitudes of the gradient loads are designed based on a laser beam spot size for the emission spectroscopy analysis and a common particle size distribution range of a target detection inclusion in the steel; and
fabricating an indentation array with a set number of rows and columns on the surface of the steel matrix sample to form a series of square pyramidal indentation micropores with different sizes, and determining a spacing between the indentation micropores based on the laser beam spot size for the emission spectroscopy analysis, such that a beam spot does not affect adjacent indentation micropore regions during laser excitation.

5. The method for preparing and calibrating a standard sample for quantitative analysis of inclusions in steel according to claim 2, wherein the powder filling and fixation of S2 comprises:
placing the steel matrix sample with the indentation micropores in the container containing the nano-oxide powder of the target element, wherein the container is a flexible rubber mold, the nano-oxide powder of the target element is a single-component powder or a multi-component composite powder, and the multi-component composite powder is prepared by weighing and uniformly mixing multiple element powders in a predetermined ratio;
preparing, if the single-component powder is filled, multiple indentation micropore samples with a series of gradient loads, and pressing nano-oxide powders of different elements thereinto respectively, to obtain multiple univariate standard samples with different concentration gradients; and
preparing, if the multi-component composite powder is filled, an indentation micropore array sample with a same load, and before the filling, weighting masses of components and calculating a ratio thereof to obtain a multivariate standard sample that can obtain concentration gradient relationship of multiple elements at one time; and
sealing the mold filled with powder, connecting to a high vacuum system for vacuuming treatment, then placing the sealed mold in a cold isostatic press, and applying a hydrostatic pressure at a room temperature and holding the pressure for a certain period of time, during which an isotropic hydrostatic pressure causes the nano-oxide powder of the target element to fill a square pyramidal cavity of each indentation micropore evenly and densely, to form a standard detection point with clear composition and controllable content.

6. The method for preparing and calibrating a standard sample for quantitative analysis of inclusions in steel according to claim 1, wherein the quantitative relationship model between indentation size and powder mass is expressed as:
$$C ( wt \% ) = \frac{{W}_{A}}{{W}_{max}} = \frac{1 / 3 \cdot {S}_{A} \cdot {h}_{A} \cdot {ρ}_{A}}{1 / 3 \cdot {S}_{max} \cdot {h}_{max} \cdot {ρ}_{A}} = \frac{{V}_{A}}{{V}_{max}}$$
wherein, W_{A} represents an absolute mass of the nano-oxide powder of the target element filled in an indentation micropore, Wₘₐₓ represents a powder mass in a largest indentation micropore, which is used as a baseline to establish relative mass ratio relationship; ρ_{A} represents a theoretical density of the nano-oxide powder of the target element; V_{A} represents a volume of the nano-oxide powder of the target element; Vₘₐₓ represents a volume of the largest indentation micropore; S_{A} is bottom area of the nano-oxide powder of the target element; Sₘₐₓ is bottom area of the largest indentation; and C(wt %) represents a content of the nano-oxide powder of the target element.

7. The method for preparing and calibrating a standard sample for quantitative analysis of inclusions in steel according to claim 1, wherein the performing emission spectroscopy analysis on the standard sample comprises:
S5, using LIBS or Spark-OES targeted analysis technology to perform positioned excitation on indentations on the standard sample to collect the intensity of the characteristic spectral line of each element, which comprises following steps:
placing the prepared standard sample in a laser-induced breakdown spectroscopy or spark-source emission spectroscopy analysis system, scanning and observing the surface of the standard sample by using a high-precision moving platform and a microscopic positioning device equipped in a system, to locate each indentation micropore region filled with the nano oxide powder of the target element;
formulating corresponding laser excitation strategies based on differences in sizes of the indentation micropores, wherein for indentation micropores with a size larger than a set value, a multi-pulse laser excitation mode is adopted; and for indentation micropores with a size smaller than the set value, a single-pulse laser excitation mode is selected; and
after excitation at each positioning point, acquiring an original characteristic spectral signal of the target element, and then processing the original characteristic spectral signal by a continuous background subtraction algorithm to eliminate matrix interference and obtain a net intensity value of the characteristic spectral line of each element at the indentation micropores.

8. The method for preparing and calibrating a standard sample for quantitative analysis of inclusions in steel according to claim 1, wherein the establishing the quantitative calibration curve between elemental content and spectral intensity comprises:
S6. establishing the quantitative calibration curve between elemental content and spectral intensity based on the powder mass and the measured net spectral intensity, which comprises following steps:
performing linear fitting in a double logarithmic coordinate system, by using the powder masses corresponding to the indentation micropores calculated based on the quantitative relationship model between indentation size and powder mass as an ordinate, and the measured intensity of the characteristic spectral line of each element at the indentation micropores as an abscissa, to obtain the quantitative calibration curve of the element.

9. A standard sample, obtained by using the method for preparing and calibrating a standard sample for quantitative analysis of inclusions in steel according to claim 1, wherein the standard sample is a univariate standard sample or a multivariate standard sample.

10. A method for quantitative analysis of multiple non-metallic inclusions in steel based on emission spectroscopy, comprising following steps:
obtaining a quantitative calibration curve between elemental content and spectral intensity by applying the method for preparing and calibrating a standard sample for quantitative analysis of inclusions in steel according to claim 1; and
performing emission spectroscopy analysis on a to-be-tested steel sample to obtain intensity of a characteristic spectral line of each element in the inclusions, and quantitatively analyzing an elemental content of the inclusions in the to-be-tested steel sample based on the quantitative calibration curve between elemental content and spectral intensity.

11. A method for quantitative analysis of multiple non-metallic inclusions in steel based on emission spectroscopy, comprising following steps:
establishing the quantitative calibration curve between elemental content and spectral intensity by applying the standard sample according to claim 9; and
performing emission spectroscopy analysis on a to-be-tested steel sample to obtain intensity of a characteristic spectral line of each element in the inclusions, and quantitatively analyzing an elemental content of the inclusions in the to-be-tested steel sample based on the quantitative calibration curve between elemental content and spectral intensity.
